(19) United States
(12) Patent Application Publication
(10) Pub. No.: US 2018/0161001 A1
(43) Pub. Date: Jun. 14, 2018

(54) ULTRASOUND SYSTEM AND METHOD OF MAKING AND USING SAME

(71) Applicant: SONACARE MEDICAL, LLC, Charlotte, NC (US)

(72) Inventors: Ralf Seip, Charlotte, NC (US); Rodrigo Chaluisan, Charlotte, NC (US); Mark Carol, Charlotte, NC (US)

(21) Appl. No.: 15/842,821

(22) Filed: Dec. 14, 2017

Related U.S. Application Data
(60) Provisional application No. 62/433,989, filed on Dec. 14, 2016.

(51) Int. Cl. A61B 8/00; A61N 7/00; A61B 8/08; G01S 7/52

(52) U.S. Cl. CPC A61B 8/4218; A61N 7/00; A61B 8/4483; A61B 8/461; A61N 2007/0052; A61B 8/5207; A61B 8/483; G01S 7/5205; A61B 8/4405

(57) ABSTRACT

An ultrasound delivery system can include a console assembly having a display and a computer system, and a positioning assembly operatively connected to the console assembly. The positioning assembly can be articulable between a plurality of different configurations upon receipt of one or more instructions from the computer system. The positioning assembly can include at least one gripper mechanism configured to selective grip and release. At least one probe assembly can include an ultrasound transducer assembly, a first bolus assembly and a second bolus assembly. The first bolus assembly can have at least a different geometry than the second bolus assembly. At least a portion of the ultrasound transducer assembly can be removably positionable within each of the first bolus assembly and the second bolus assembly. At least a portion of the probe assembly can be selectively connected to gripper mechanism and moved by the positioning assembly.

### CROSS-REFERENCE TO RELATED APPLICATION

This application claims priority to U.S. Provisional Application No. 62/433,989, filed Dec. 14, 2016 and titled “Robotic Therapeutic Ultrasound System and Method,” which is herein incorporated by reference.

### FIELD

In one embodiment, the presently disclosed technology relates generally to robotic control of one or more ultrasound transducers. More particularly, one embodiment of the presently disclosed technology relates to an ultrasound system with one or more interchangeable parts.

In one embodiment, the presently disclosed technology relates generally to debubbling an ultrasound probe. More particularly, one embodiment of the presently disclosed technology relates to automatically or robotically removing gas and/or trapped gas bubbles from a bolus assembly of a high intensity focused ultrasound (HIFU) system.

### BACKGROUND

It is known to use therapeutic ultrasound in a clinical setting for the treatment of a multitude of diseases and conditions in a non-invasive or minimally invasive manner. One such example is described in U.S. Patent Application Publication No. 2014/0243677. In operation, it is necessary to physically or mechanically translate the ultrasound transducer assembly to reach a patient and/or target a particular volume of tissue of the patient. Due at least in part to the multitude of procedures in which therapeutic ultrasound can be beneficial, different procedures typically require the development of an application-specific probe assembly, which includes a transducer assembly, an acoustic coupling bolus assembly, mechanical positioning actuators, and corresponding control electronics. In contrast, transducer assemblies alone (which form part of the probe and are housed inside the bolus assembly) could be used to deliver ultrasound energy in a variety of instances. While effective, such prior art application-specific probe assembly designs limit the usability of the probe assembly to specific applications, thereby delaying the deployment of the technology to other applications.

Prior art transducer positioning also tends to be application-specific. The specific medical procedure dictates the speed and extent of motion required for the ultrasound transducer to reach all or desired target tissues that are part of a therapeutic ultrasound treatment. In many prior art cases, the probe assembly houses mechanical actuators (such as motors) that aid with positioning the transducer on the target, and/or manipulate the position and orientation of the transducer so as to be able to deliver therapeutic ultrasound energy to a larger target volume. Mechanical transducer translation within the probe assembly can be further augmented with electronic beam steering to target a larger volume. In all of these cases, the therapeutic transducer or transducer assembly is tightly linked and coupled to the bolus assembly. Such an arrangement produces probes or probe assemblies that are excellent for a single or particular application, but have restricted use for other applications, thereby limiting the usability or applicability of a particular probe assembly.

In addition, dissolved gases and/or gas bubbles within an ultrasound propagation path disrupt the ability of an ultrasound system to deliver energy to the target tissue of the patient. Gas bubbles may appear during the routine system preparation step, when the probe assembly or the bolus assembly and associated fluid path is primed and filled with the coupling fluid needed to transfer the ultrasound energy from the face of the transducer, through the coupling fluid, and into the patient's target tissue. It is known to attempt to at least partially remove bubbles (e.g., air bubbles) from the bolus assembly, but prior art debubbling processes are time consuming, though often necessary.

### SUMMARY

Embodiments of the presently disclosed technology overcome certain drawbacks of prior art designs and satisfy the above-outlined and other objectives.

In one embodiment, the presently disclosed technology is directed to a system having a transducer assembly, an acoustic coupling bolus, and one or more mechanical positioning components that are separable from and interchangeable with each other. These elements can be coupled together and substituted in multiple ways, which significantly expands the application space and flexibility of the system, without requiring different or unique probes for each different application.

### BRIEF DESCRIPTION OF THE DRAWINGS

The foregoing summary, as well as the following detailed description of the presently disclosed technology, will be better understood when read in conjunction with the appended drawings. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 is a perspective view of an ultrasound system according to one or more embodiments of the presently disclosed technology, wherein two separate probe assemblies are shown;

FIG. 2 is a perspective view of a console and a coupling fluid system management system according to one embodiment of the presently disclosed technology;

FIG. 3 is a perspective view of a positioning system according to one embodiment of the presently disclosed technology;

FIG. 4 is a perspective view of at least a portion of a transducer assembly according to one embodiment of the presently disclosed technology;

FIG. 5 is a top plan view of a bolus assembly according to one embodiment of the presently disclosed technology;

FIG. 6 is a perspective partially exploded view of a probe assembly according to one embodiment of the presently disclosed technology;

FIG. 7A is a perspective view of a proximal end of one embodiment of a transducer assembly of one embodiment of the presently disclosed technology;

FIG. 7B is a perspective view of a proximal end of another embodiment of the transducer assembly of the presently disclosed technology;

FIG. 7C is a perspective view of a proximal end of yet another embodiment of the transducer assembly of the presently disclosed technology; and

FIG. 8 is a schematic diagram of a computing system of one embodiment of the present disclosure.

### DETAILED DESCRIPTION

While systems, apparatus and methods are described herein by way of examples and embodiments, those skilled in the art recognize that the systems, apparatus and methods of the presently disclosed technology are not limited to the embodiments or drawings described. It should be understood that the drawings and description are not intended to be limited to the particular form disclosed. Rather, the presently disclosed technology covers all modifications, equivalents and alternatives falling within the spirit and scope of the appended claims. Any headings used herein are for organizational purposes only and are not meant to limit the scope of the description or the claims.

As used herein, the words “is” and “may” are used in a permissive sense (i.e., meaning having the potential to) rather than the mandatory sense (i.e., meaning must). Similarly, the words “include,” “including,” and “includes” mean including, but not limited to. Unless specifically set forth herein, the terms “a,” “an” and “the” are not limited to one element but instead should be read as meaning “at least one.” The terminology includes the words noted above, derivatives thereof and words of similar import.

As described below, various embodiments of the presently disclosed technology may be readily combined or even omitted. While the presently disclosed technology is described with reference to ultrasound or HIFU, the presently disclosed technology is not so limited and has applicability to other fields and uses. Furthermore, like elements among different embodiments are identified with one or more prime symbols (′) after like reference numerals. Description of similar or identical features between the embodiments may be omitted herein for the sake of brevity and convenience only.

One way to solve the drawback(s) of the prior art (e.g., increase the applicability or usability of probe assemblies) is to de-couple or separate the transducer assembly from the probe assembly (which includes the bolus assembly and the transducer positioning system), which can significantly increase the flexibility or usability of the therapeutic system. For example, the interchangeability of the presently disclosed technology could allow a particular number of bolus assemblies (e.g., twenty five different bolus assemblies) be used with a more limited number of transducer assemblies (e.g., four different transducer assemblies). Since transducer assemblies tend to be more complicated and expensive than bolus assemblies, this interchangeability can reduce costs of designing and manufacturing an ultrasound system.

In particular, the system 10 of the presently disclosed technology allows for the easy substitution of any of the three (or more) involved subsystems: (1) at least one therapeutic and/or imaging ultrasound transducer assembly 16, 16′, (2) at least one acoustic coupling bolus or bolus assembly 18, 18′, and (3) at least one positioning system 20, 20′, with alternate implementations, enabling the realization of a platform technology for general-purpose therapeutic ultrasound delivery. De-coupling the three (or more) subsystems also allows for the implementation of a disposable bolus, as this piece no longer needs to be an integral or inseparable part of the probe assembly itself. In one embodiment, de-coupling the three (or more) subsystems enables different transducer assemblies (which could be application-specific) to share a common positioning system (such as a general-purpose robotic positioner, for example), which expands the application space of the therapeutic ultrasound system.

1. Console Assembly

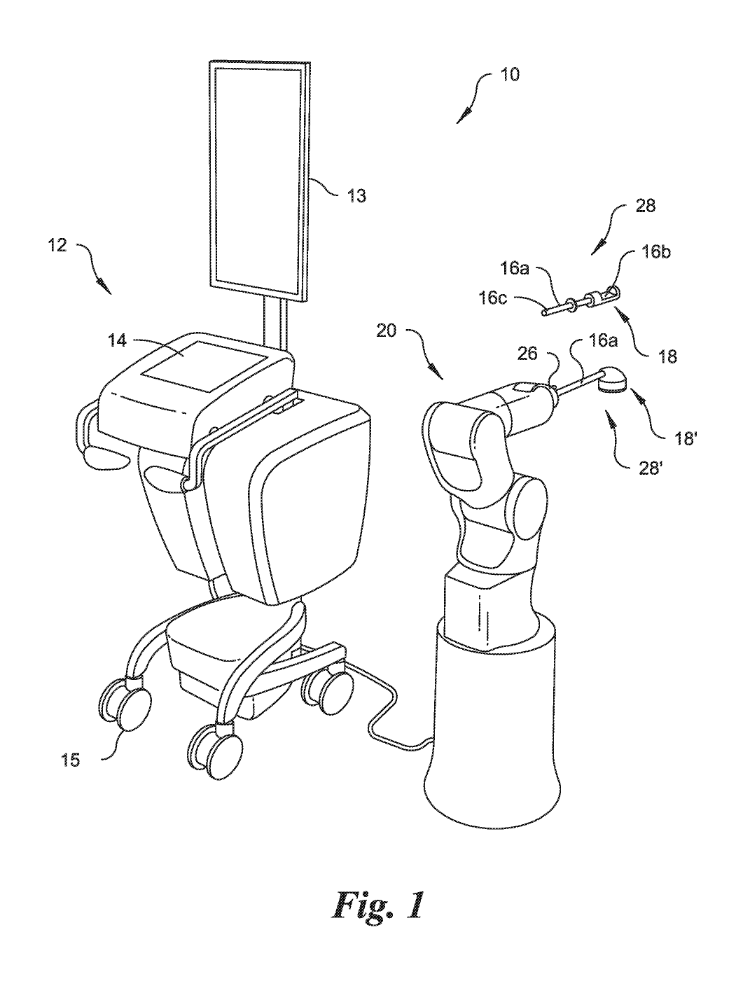

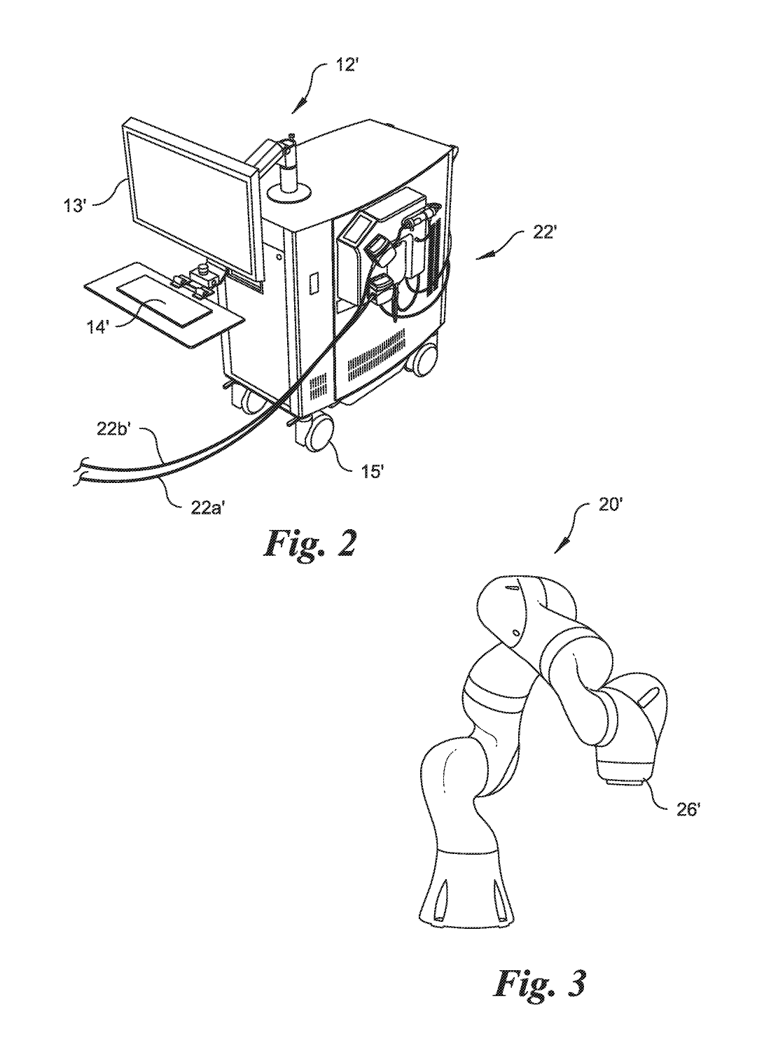

Referring to FIGS. 1 and 2, in certain embodiments, the system 10 includes a console assembly 12, 12′ that is aligned, controls or interacts with and support one or more of the above-described subsystems. In particular, the console assembly 12, 12′ is able to drive any or all remaining subsystems that are connected to it. Optionally, the console assembly 12, 12′ can include, support or house a display or monitor 13, 13′, a keyboard or other input device 14, 14′, one or more wheels 15, 15′, and/or one or more computing devices, as described in detail below. The display can include a touchscreen, a webcam, a speaker and a microphone. The keyboard can be a touchscreen and water-resistant, and can include a trackball or other control features.

The console assembly 12, 12′ can be relatively small (e.g., a small footprint) or compact, and easily movable within an operating or medical examination room. The details to enabling such a platform/general purpose approach lie in the definition of the various interfaces between the individual subsystems themselves, and between the subsystems and the console assembly 12, 12′. This document describes these interfaces, together with how to use such a system in clinical practice.

In one embodiment, the one or more transducer assemblies 16, 16′, the one or more bolus assemblies 18, 18′, and the one or more positioning assemblies 20 can be mixed and/or interchanged as needed for a specific application. In such a system and in one embodiment, the console assembly 12, 12′ can be the only device that is shared among all of these sub-assemblies, and the only piece that is common across all applications.

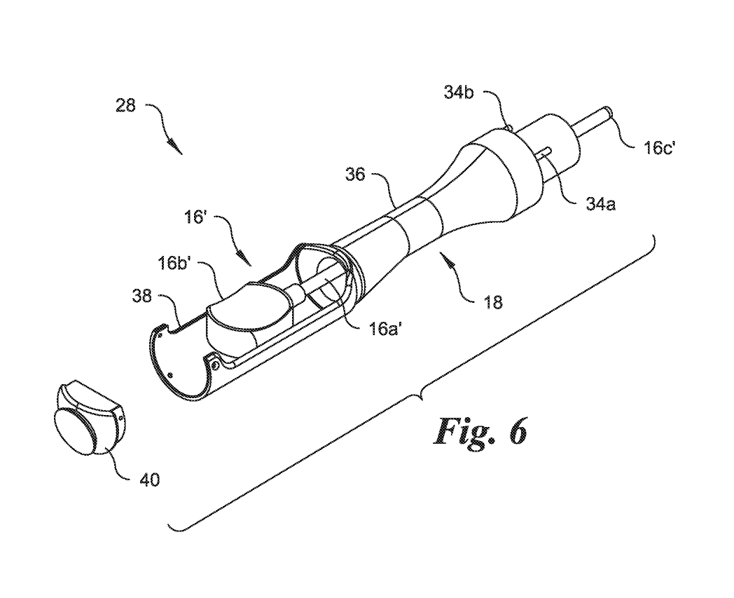

FIG. 2 shows a therapeutic ultrasound coupling fluid management system 22′ attached to and/or support by the console assembly 12′. The fluid management system 22′ can therefore be located exterior to a housing of the system assembly 12′ (as in FIG. 2) or be located within a housing of the console assembly 12 (as in FIG. 1). In the former embodiment, the fluid management system 22′ can be removable from the console assembly. In the latter embodiment, the fluid management system can be a non-removable part of the console assembly itself. Optionally, the fluid management system can be integrally connected or attached to the probe assembly or be attached to or part of the positioning assembly. The fluid management system 22′ can be a closed, single-loop configured to circulate fluid (e.g., sterile water) within, to and from the one or more bolus assemblies 18 and/or one or more transducer assemblies. Optionally, the fluid management system 22′ can include tubing 22a′, 22b′ (see FIG. 2) that is configured to fluidly connect to fluid ports (e.g., a fluid inlet port 34a and a fluid outlet port 34b; see FIG. 6) of the bolus assembly 18 or one or more fluid ports of the transducer assembly. In one embodiment, the console assembly controls the coupling fluid management system, as this forms an integral part of the system concept described in this disclosure, enabling automated fluid path priming, bolus assembly priming, and transducer assembly positioning.

In one embodiment, the console assembly can execute the system setup, treatment planning, and treatment execution steps. Optionally, the console assembly can identify and control the positioning assembly, and the console assembly can identify and control the transducer assembly. In one embodiment, via setup files or other software, the console assembly will know the capabilities, degrees of freedom, and other characteristics of the positioning system when it is connected to it (via Ethernet, for example). In one embodiment, via setup files or other software, the console will also know the capabilities, configuration, and other characteristics of the transducer assembly when it is connected to it. In one embodiment, the console assembly will not contain any positioning electronics and/or hardware, as all of these items are a part of the positioning assembly, thus providing the flexibility to connect different positioning assemblies to the console, depending on the application.

Optionally, the console assembly contains at least one ultrasound imaging subsystem (including, for example, at least one pulser/receiver, beamformer, power supply, and associated control electronics—which connects to at least one ultrasound imaging array located in the transducer assembly), and at least one ultrasound therapy subsystem (including, for example, at least phasing electronics, power supply, and associated control electronics—which connects to at least one ultrasound therapy array located in the transducer assembly). In some cases, the console assembly will also contain a single channel RF amplifier, to be connected to the transducer assembly when it contains single-element transducers (rather than arrays) for enabling the ultrasound therapy function.

In one embodiment, the ultrasound therapy subsystem includes a large amount of low-power amplifiers (128-1024 typical). These allow for system flexibility when connecting various ultrasound therapy arrays to the console. It is envisioned that not all amplifiers will be used for all transducer assemblies. For example, one transducer assembly can contain a 64-element imaging array, and a 256-element therapy array. In this case, the imaging array is connected to the ultrasound imaging subsystem via its own connector, and the therapy array is connected to the therapy subsystem via its own connector(s), however, only 256 amplifiers (out of 1024) of the therapy subsystem are used to control the functions of the therapy array.

2. Positioning Assembly

FIGS. 1 and 3 show two embodiments of a positioning assembly 20, 20′ of the presently disclosed technology. Each positioning assembly 20, 20′ can be operated or controlled by a user (e.g., a doctor, nurse or technician) through the console assembly 12, 12′. In certain embodiments, the positioning assembly 20, 20′ is an articulating, mechanical, robotic arm. In another embodiment, the positioning assembly is a multi-axis and multi-degree-of-freedom robotic arm. The positioning assembly 20, 20′ can be connected to the console assembly 12, 12′ in any of a variety of manners, such as through a wire connection (see FIG. 1) or wirelessly, via a network interface, USB interface, or other suitable interface.

One end of each positioning assembly 20, 20′ may be positioned on or supported by a support structure (e.g., a ground surface, a table surface, or a stand, such as that shown in FIG. 1, or include attachment mechanisms (e.g., one or more clamps) that allow the positioning assembly to be attached to an operating room table) and a second end of each positioning assembly 20, 20′ can be attached or connected to the probe assembly. More particularly, in some embodiments, a free or movable end of the positioning system 20, 20′ can be attached or connected to one or more of the transducer assemblies 16, 16′ and/or the bolus assemblies 18, 18′.

In one embodiment, the positioning assembly 20, 20′ can include at least one and possibly two or more mating connectors or gripper mechanism 26, 26′ that is compatible or matable with the attachment mechanism of either or both the bolus assembly and the transducer assembly. For example, the free or second end of the positioning assembly can be configured to grab or connect to the bolus assembly 18, 18′ and/or the transducer assembly 16, 16′ securely at this point, so as to be able to position, place and/or manipulate either one or both of the bolus and transducer assemblies for their use in the delivery of the ultrasound therapy or other procedure. Each gripper mechanism 26, 26′ can be in any of a variety of forms. For example, one or more of the gripper mechanisms 26, 26′ can include a rotatable nut or drill chuck that can be turned (e.g., clockwise) to engage and/or grab a portion (e.g., the shaft and/or interconnect mechanism) of the transducer assembly and/or the bolus assembly. Alternatively, the nut or drill check can be moved linearly toward or away from the positioning assembly 20, 20′ to loosen or tighten, respectively the gripper mechanism 26, 26′. In another embodiment, one or more of the gripper mechanisms 26, 26′ can include two or more fingers that can be selectively moved toward or away from a portion (e.g., the shaft and/or the interconnect mechanism) of the transducer assembly and/or the bolus assembly. Optionally, one or more of the gripper mechanisms 26, 26′ can include magnets to create or facilitate the attachment, one or more slotted receptacles and corresponding mating surfaces, a spring-loaded “push-and-turn” action mechanism, a “click-and-latch” mechanism, a thumbscrew attachment mechanism, or the like. These connections can be motorized, or can be manually actuated by the user.

In one embodiment, the free or second end of the positioning assembly includes or utilizes only a single gripper mechanism to grasp at least a portion of the transducer assembly 16, 16′. In such an embodiment, the bolus assembly 18, 18′ can be movable with respect to the transducer assembly 16, 16′, which in turn can be controlled or manipulated by the positioning system 20, 20′. In other words, in one embodiment, the bolus assembly 18, 18′ can be loosely attached to a shaft of the transducer assembly 16, 16′. Thus, in such an embodiment, the bolus assembly 18, 18′ is not and cannot be fixedly attached to the transducer assembly 16, 16′. Thereby, the positioning assembly 20, 20′ will only be able to perform or effectuate certain movements of the transducer assembly 16, 16′ without displacing the bolus assembly 18, 18′ from its original position on or within the patient (which is not desired).

Optionally, the system 10, 10′ can employ one or more imaging transducers (e.g., of or on the transducer assembly) to identify one or more portions (e.g., interior) or edges of the transducer assembly 18 and create a map or coordinate system of the position, configuration and/or location of the transducer assembly 18, 18′ to inform the user and/or the computer system of the console assembly 12, 12′. Thus, by utilizing the ultrasound imaging function of the transducer assembly 16, 16′, for example, the system 10, 10′ can image, probe and/or detect the initial position of the transducer assembly 16, 16′ within and/or with respect to the bolus assembly 18, 18′. In addition, in one embodiment, during this registration step, the transducer assembly 16, 16′ can build up a 2D or 3D map and orientation of the bolus assembly 18, 18′, such as an inside cavity thereof, and the location of the transducer assembly 16, 16′ within it by displacing the transducer assembly 16 by small steps initially to probe or test-out its position and orientation within the bolus assembly 18, 18′.

In one embodiment, the inside geometry of the bolus assembly 18, 18′ is known to the system 10, 10′ prior to beginning a medical procedure. Ultrasound edges and boundaries detected by the ultrasound imaging function of the transducer assembly 16, 16′ can then be matched up with the known inside geometry of the bolus assembly 18, 18. This information can then be used to determine appropriate transducer assembly 16, 16′ orientation and limits of motion of the positioning assembly 20, 20′ and the attached transducer assembly 16, 16′, so that displacement of the bolus assembly 18, 18′ with respect to the patient does not occur during subsequent ultrasound therapy delivery, via appropriate coordinate system transformations. In one embodiment, these transformations would map the newly determined positioning assembly's coordinate system onto the transducer assembly's coordinate system (which is directly registered to the patient's coordinate system via the fixed bolus assembly), which is the most intuitive from a treatment planning, monitoring, and delivery approach.

In one embodiment, the positioning assembly 20, 20′ can hold the bolus assembly 18, 18′ firmly in place while the user attaches the transducer assembly 16, 16′ to the bolus assembly 18, 18′. In this sense, the positioning system 20, 20′ provides a third or “helping” hand, which assists the user during system 10, 10′ preparation.

In another embodiment, the positioning assembly 20, 20′ can hold the bolus assembly 18 and the transducer assembly 16, 16′ firmly in place while the user completes the assembly of the bolus assembly 18, 18′, and then dresses the bolus assembly 18, 18′ with an acoustically transparent sheath (if necessary).

Optionally, the positioning assembly 20, 20′ can hold the transducer assembly 16, 16′ and the bolus assembly 18, 18′ in place while the user connects one or more fluid paths of the fluid management system 22′ to the bolus assembly 18, 18′, connects an interconnect of the transducer assembly 18 to the console assembly 12, 12′, and/or primes the fluid path(s) of the fluid management system 22′.

In one embodiment, the positioning assembly 20, 20′ can place the bolus assembly 18, 18′ and the transducer assembly 16, 16′ at a correct or desired location on the patient for the ultrasound therapy, thereby allowing the user to attach the bolus assembly 18, 18′ to the target tissue (if needed in some extracorporeal applications), or hold the bolus assembly 18 in a particular position (such as inside the patient). This positioning may occur automatically (e.g., via optical guidance, RF/IR local positioning feedback, etc.) or user guided (e.g., by manually placing the bolus assembly and the transducer assembly on the patient while it is still connected to the positioning assembly, via one or more joysticks, pushbuttons, or the like). In one embodiment, the positioning assembly 20, 20′ can debubble the bolus assembly 18, 18′ automatically, as described below.

In one embodiment, the positioning assembly 20, 20′ can attach to the transducer assembly 16, 16′ and translate, rotate, tilt or otherwise move the transducer assembly 16, 16′ to position and manipulate the transducer assembly within or with respect to the bolus assembly. Such movement allows the system or the user to orient and translate the transducer to deliver ultrasound energy to the target tissue, as defined by the user and/or treatment plan. Furthermore, by incorporating ultrasound image guidance from the transducer assembly 16, 16′ (or other image guidance approaches), the positioning system 20, 20′ can also in real-time track and compensate for transducer/target tissue relative motion, so that the ultrasound could be continually delivered to the same target location, independent of tissue motion.

In one embodiment, because the positioning assembly 20, 20′ is a subsystem of the entire ultrasound system 10, it may be switched out and replaced by a different positioning assembly, as required by the application and/or determined by the user. This way, if an application or medical procedure requires high-positioning accuracy and a small payload capacity, a custom positioning assembly can be used, and replaced with a different one if an application requires a small degree of motion but a high payload capacity, for example. The intent of the positioning assembly is to be able to replicate any and all functions that a custom and application-specific probe would perform with its transducer, but in a much more flexible way.

3. Probe Assembly

As described above, the probe assembly 28, 28′ can include a transducer assembly 16, 16′ and a bolus assembly 18. As shown in FIG. 1, the system 10 can utilize more than one probe assembly, which decreases costs and increases the functionality and usability of the system 10. For example, the probe assemblies 28, 28′ shown in FIG. 1 can utilize the same transducer assembly, while utilizing a different bolus assembly.

Referring to FIGS. 4 and 6, the transducer assembly 16, 16′ can include one or more of the following components: a shaft 16a, 16a′ having at least one transducer 16b, 16b′ (e.g., imagining and/or therapy) at a first or distal end thereof, and an interconnect mechanism 16c, 16c′ at a second or opposing proximal end thereof. The transducer can include one or several ultrasound transducers or ultrasound arrays (e.g., 2D or 3D), which can carry-out the ultrasound imaging and/or therapy function of the particular application. The transducer(s) 16b, 16b′ can deliver either or both ablative and non-ablative therapy. The therapeutic transducers could be single-element transducers, spherically-focused devices, cylindrically focused devices, phased arrays (e.g., for imaging and therapy delivery), or any other structure used for delivering ultrasound therapy. The imaging transducers could be single-element transducers, linear arrays, sector arrays, or any other structure used for ultrasound imaging. The transducer could also be comprised of dual-mode devices, which are capable of being used for both ultrasound imaging as well as therapeutic applications. In one embodiment, the transducer can include one or more other sensors, such as temperature sensors, infrared (IR) sensors, optical sensors, radio frequency (RF) sensors or the like, if needed for delivering or controlling the ultrasound therapy.

Optionally, the transducer(s) can incorporate electronics (such as multiplexers, impedance matching, or micro-beamformers) that allow for simplifying and reducing the number of leads/connections on the transducer interconnect side, so to facilitate connecting the transducer assembly to its driving electronics. The transducer(s) can be any size or shape required by the application, and can be larger (in terms of thickness, for example) than the shaft itself. In one embodiment, the transducer assembly can include one ultrasound imaging array, one ultrasound therapy array, and one thermistor.

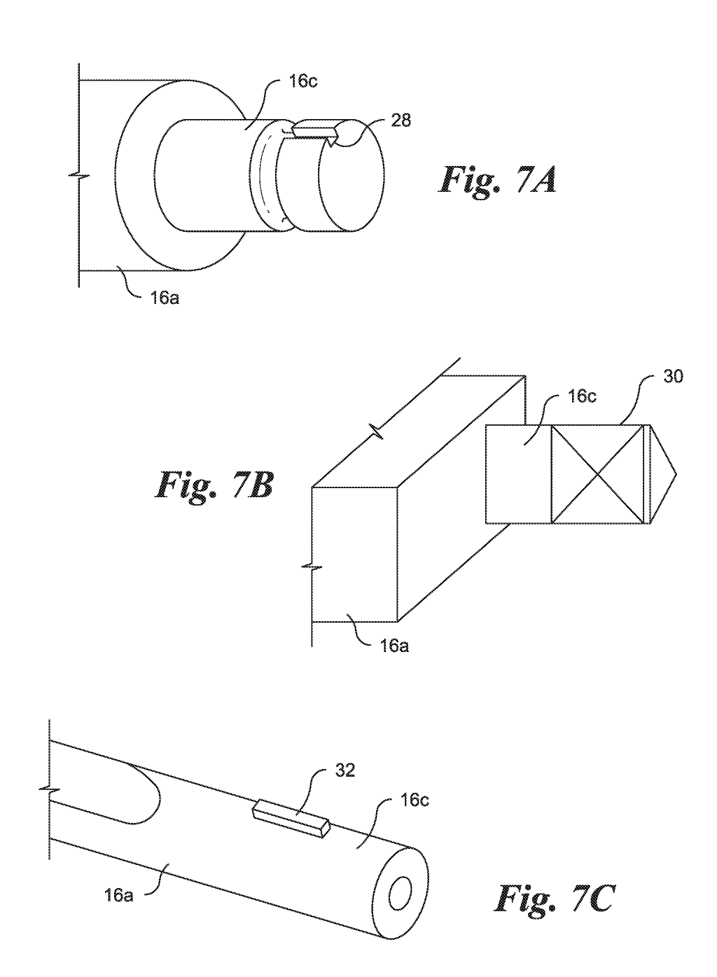

In one embodiment, at least a portion of or the entire shaft 16a is larger in diameter than the interconnect mechanism 16c of the transducer assembly 16 (see, e.g., FIG. 7A). Such a configuration can facilitate mating, attaching, and/or connecting the transducer assembly 16 with the bolus assembly 18, 18′ and/or another component, as described below.

The interconnect mechanism 16c, 16c′ can electrically connect the transducer assembly 16, 16′ to the console assembly 12, 12′. In one embodiment, the interconnect mechanism 16c, 16c′ can be protected by a removable tip or cap when the transducer assembly 16, 16′ is placed inside or inserted into the bolus assembly 18, 18′. The tip or cap can protect one or more electrical contacts of the interconnect mechanism 16c, 16c′ from fraying, bending and or breaking, or from being covered with lubrication during the insertion process, for example. The electrical contacts can connect the transducer elements/arrays/crystals to an outside amplifier/controller, together with any of the other electrical connections that typically need to be made (i.e., connect to an embedded EEPROM or similar, which contains probe serial number information, calibration information, and the like),

In one embodiment, when the interconnect mechanism 16c, 16c′ is exposed, its entry into and/or attachment to the shaft 16a, 16a′ is fluid-tight. Optionally, electrical interconnection of the transducer assembly 16, 16′ is envisioned to be either performed by the user when preparing the system 10, 10′ for use, or may be accomplished automatically and be part of the positioning system 20, 20′ when it or its gripper mechanism 26, 26′ attaches to the shaft 16a and/or the interconnect mechanism 16c, 16c′.

In one embodiment, the transducer assembly 16, 16′ includes or contains an identifier, so as to uniquely identify it to the console assembly 12, 12′, so that appropriate software or drivers can be loaded and used to drive or control the transducer assembly 16, 16′. Such an identifier can be in the form of a non-volatile memory located within the transducer assembly (e.g., EEPROM, RFID tag, etc.) that contains a serial number or other unique classification information, a barcode on the transducer shaft 16a, 16a′ or other identification means.

In one embodiment, the transducer assembly is a single-use apparatus. In another embodiment, the transducer assembly is a multiple-use apparatus, where it is designed to be repeatedly sterilisable or cleanable, as required by the application. In one embodiment, the transducer assembly is a subsystem of the entire ultrasound system. Thus, the transducer assembly can be selectively switched out and replaced by a different transducer assembly, as required by the application.

Referring to FIG. 6, in one embodiment the bolus assembly 18 can be configured to accept, receive and/or at least partially house the transducer assembly 16, 16′ (e.g., via or utilizing one or more fluid-proof and flexible seals). For example, in one embodiment, the shaft 16a, 16a′ of the transducer assembly 16, 16′ interfaces and/or connects to the bolus assembly 18. The connection or contact between the shaft of the transducer assembly and the bolus assembly can form a flexible but fluid-tight seal, which allows the transducer assembly to freely move (e.g., translate, angulate, and/or rotate) within or with respect to the bolus assembly, and/or allows the bolus assembly to be filled with the coupling fluid required to acoustically couple the transducer assembly to the target tissue.

Optionally, the bolus assembly 18, 18′ can be disposable. In one embodiment, one or more inlet and/or outlet ports of the bolus assembly 18 can be connected to the coupling fluid management system 22′. In one embodiment, the transducer and/or the entire transducer assembly is fluid-tight, as during operation it will be fully surrounded by a coupling fluid (e.g., water) entering and leaving the bolus assembly 18, 18′.

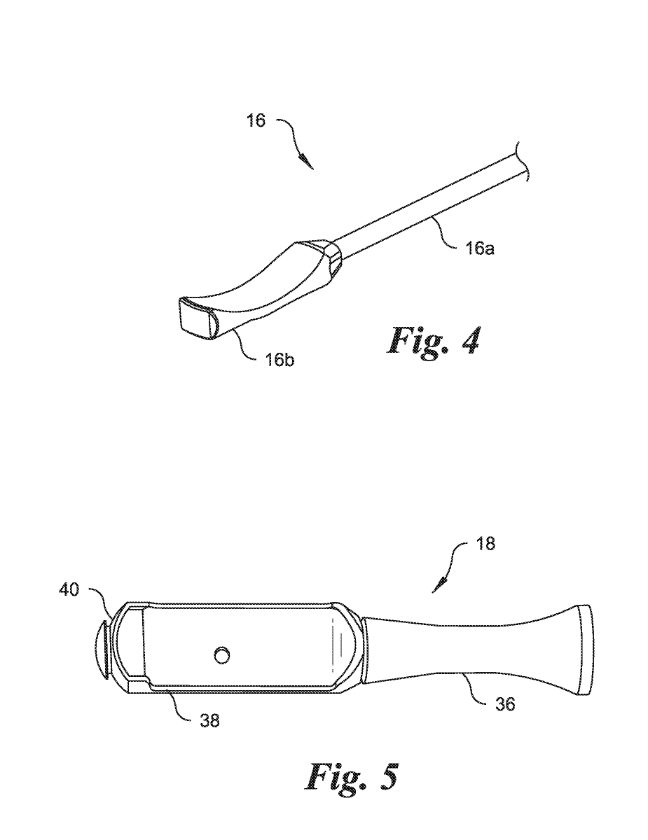

As shown in FIGS. 5 and 6, in one embodiment, the bolus assembly 18 can be sized, shaped and/or configured to have a relatively narrow or tapered neck portion 36, thereby allowing it to be placed trans-rectally or trans-vaginally in intracavity applications without external attachment mechanisms. Alternatively or additionally, the bolus assembly 18, 18′ can be employed for extracorporeal use and/or laparoscopic use, for example. The bolus assembly 18 can have a window 38 located near a cap 40 at a distal end of the bolus assembly 18. The window 38 can be covered by an acoustically transparent and distendable membrane that can be bonded to at least a portion of a housing of the bolus assembly 18 that surrounds the window 38. Optionally, the membrane can be fluid impermeable. Optionally, the cap 40 can be selectively removable from a portion of the housing of the bolus assembly 18 (see FIG. 6).

The transducer assembly 16, 16′ can include one or more attachment mechanisms to allow it to be more easily attached to the positioning system 20, 20′ and/or placed proximate to the target region on a patient (for extracorporeal applications). More particularly, in one embodiment, a proximal tip or end of the shaft 16a, 16a′ of the transducer assembly 16 includes one or more spaced-apart keyed attachment mechanisms. Through the attachment mechanism(s), the transducer assembly 16, 16′ can be connected to and manipulated by the positioning system 20, 20′ to execute the ultrasound therapy and/or imaging procedure. Thus, in one embodiment, the attachment mechanism(s) can mechanically connect the transducer assembly 16, 16′ to the positioning system 20, 20′.

For example, as shown in FIGS. 7A-7C, the transducer assembly 16 can contain one or more keyed attachment points or structures, which enables the positioning assembly 20, 20′ to connect to and manipulate, position and/or place the transducer(s) 16b and/or the transducer assembly 16 on the patient or on the target tissue. In one embodiment, the attachment mechanism of the transducer assembly 16 provides complementary keyed attachments that incorporate translational as well as rotational stability, such as the examples shown in FIGS. 7A-7C. In one embodiment, the positioning assembly 20, 20′ can easily and quickly attach and detach from the transducer assembly 16. Optionally, various mechanisms can be used to create the quick attachment/detachment features, such as a gripper mechanisms, a magnetic mechanism, and the like.

FIG. 7A shows a groove 28 extending parallel to a longitudinal axis of the shaft 16a of the transducer assembly 16. In another embodiment, one groove can be located on one side or surface of the shaft 16a, and an identical or similar groove can be located on an opposing side or surface of the shaft 16a. In further embodiment, multiple (e.g., circumferentially) spaced-apart grooves may be located on the shaft 16a.

FIG. 7B shows a pyramid structure 30 extending outwardly from the proximal end of the shaft 16a. In another embodiment, one groove can be located toward a first or left side of the proximal end of the shaft 16a, and an identical or similar groove can be located on a second or right side of the proximal end of the shaft 16a. In such a configuration, the two pyramid structures can be laterally spaced-apart and extend in parallel.

FIG. 7C shows a single raised key or projection 32 extending upwardly and/or outwardly from a surface of the shaft 16a. In another embodiment, one key can be located on one side or surface of the shaft 16a, and an identical or similar key can be located on an opposing side or surface of the shaft 16a. In further embodiment, multiple (e.g., circumferentially) spaced-apart keys may be located on the shaft 16a. Optionally, each key can extend parallel to the longitudinal axis of the shaft 16a.

An attachment mechanism of the bolus assembly 18, 18′ may or may not be shared with (e.g., be identical or similar to) the attachment mechanism of the transducer assembly. In one embodiment, the attachment mechanism of the bolus assembly is separate and distinct from the attachment mechanism that can connect the transducer assembly to the positioning assembly, so that both of these subassemblies may be manipulated separately (and serially) by a single positioning system, or in parallel using two positioning systems. For example, in one embodiment, one attachment mechanism can be used to hold and/or manipulate the bolus assembly, and a second or separate attachment mechanism can be used to hold and/or manipulate the transducer assembly.

4. Automatic Acoustic Coupling Bolus Debubbling

Bubbles (e.g., air bubbles) can be at least partially removed from the bolus assembly 18 and/or the transducer assembly in a number of ways. For example, bubbles can be removed by circulating the coupling fluid through the fluid path of the fluid management system 22′, by actively degassing the fluid in the fluid path, and/or by manipulating (e.g., mechanically) the probe assembly 28 and/or the bolus assembly 18 in such a way as to facilitate the air to be released from its crevices and exiting the bolus via its fluid outlet port. Such manipulation can include probe/bolus tapping (e.g., to unlodge trapped bubbles), shaking, re-orienting the probe/bolus assembly (to allow bubbles to float up to the fluid outlet port to be removed by the circulation action of the fluid path pump(s)), and/or a combination and repetition of the above manipulations. If the probe assembly or the bolus assembly also contains a mechanically-scanned ultrasound transducer assembly, it is typically also exercised within the bolus assembly, to free up and remove air bubbles and pockets that may also be potentially attached to the transducer assembly shaft, or be located in similar (and otherwise inaccessible) locations. This process is referred to as debubbling.

In one embodiment, debubbling can be automated and/or performed independently by the system prior to starting with the ultrasound therapy, thereby allowing the clinical technician or the clinician to invest their time in other necessary pre-procedure activities. More particularly, in one embodiment, the console assembly is capable of controlling both the positioning assembly and the fluid management system to execute a debubbling procedure and algorithm.

One embodiment includes the positioning assembly, with the probe assembly and/or the bolus assembly attached to its distal end (via, e.g., the gripper mechanism(s) 26, 26′) configured to manipulate the bolus assembly in such a way as to implement the debubbling step(s) described above. For example, the positioning system 20, 20′ can shake, flip, rotate, tap, etc. the bolus assembly 18 and/or the transducer assembly to remove all of the gases or bubbles trapped in the bolus assembly and the fluid path. The positioning assembly can work in concert with the fluid management system, by issuing commands to turn the circulation pump(s) on/off, rotate them at different speeds, and/or reverse them (to reverse the flow of the coupling fluid within the circulation fluid path), all automatically and under algorithm control until all tapped bubbles are removed.

The following describes the flow of actions in one embodiment of the disclosed debubbling process. First, one or more circulating fluid pumps of the fluid management system 22′ can be turned “ON.” Next, the bolus assembly 18 and/or the transducer assembly can be tapped or connected to another structure to dislodge or begin to disclose gases or bubbles. The bolus assembly and/or the transducer assembly can be oriented so that the fluid outlet port is located at a top of the bolus assembly and/or the transducer assembly, thereby allowing dislodged bubbles to float up close to the outlet port and be removed by the circulating fluid pump(s). The bolus assembly and/or the transducer assembly can be reoriented in a different position, and tapped again to dislodge bubbles. The above process can be repeated any number of times, as desired or determined by the user. Optionally, the flow of the circulating fluid pump(s) can be reversed and the above-described process can be repeated.

In one embodiment, during the above-described proposed, the presence of bubbles can be continuously monitored (as described in detail below) until no bubbles or a certain number of bubbles are detected after a certain period of time (e.g., 1 or 2 minutes). If a bubble or a certain number of bubbles are detected within the bolus assembly, the fluid path and/or the transducer assembly within the predetermined time period, the above-described process of tapping can be repeated. Optionally, the transducer assembly can be moved within the probe and/or the bolus assembly to dislodge bubbles attached or clinging to the transducer shaft. This process can be repeated until bubbles are no longer detected, only a certain number of bubbles are detected, and/or after a certain period of time has elapsed. Optionally, the system can inform the user of a successful and/or an unsuccessful debubbling process.

As mentioned above, in one embodiment, possibly via the interface to the bolus assembly, the positioning assembly and/or the system will be aware of the configuration, geometry and/or orientation of the probe and/or the bolus assembly. Optionally, this information will be pre-stored in the system software. This information can be used by the positioning system to determine how the bolus assembly and/or the transducer assembly needs to be angulated and manipulated to remove the bubbles lodged in it.

Debubbling, progression of debubbling, and completion of debubbling can be monitored and determined in any of several ways. For example, debubbling can be monitored using ultrasound-based or optical bubble detectors attached to the fluid path line(s). When bubbles stop appearing in the return fluid path from the bolus assembly for a certain amount of time, the system could indicate that the debubbling step is complete. Optionally, optical detectors can employ a LED/detector combination mounted on opposite sides of one or more tubes (e.g., the fluid path of the fluid management system). Bubbles flowing through the tube(s) would disrupt the optical signal, thus indicating the presence of bubbles. In contrast, ultrasound-based detectors can employ an ultrasound transmitter and an ultrasound receive mounted on opposite sides of a tube. Bubbles flowing through the tube would disrupt the acoustic signal, thus indicating the presence of bubbles in the tube.

In another embodiment, debubbling can be monitored using the ultrasound imaging function of the transducer assembly located in the bolus assembly. When the transducer detects that bubbles are no longer circulating within the bolus assembly (or that only a minimum level of bubbles are circulating), which can be accomplished by analyzing the ultrasound image generated by the ultrasound imaging transducer, the system can indicate that the debubbling step is complete. Optionally, ultrasound-imaging-based detectors can use ultrasound imaging arrays, which can be mounted next to a tube, or can be positioned to look into a fluid reservoir (e.g., such as a fluid reservoir of the fluid management system). These detectors can use pulse-echo techniques and generate a 2D or 3D ultrasound image of the object in front of it, including the fluid with/or without bubbles. Bubbles tend to show up as bright and easily-identifiable features in the 2D and 3D ultrasound image, thus also allowing bubbles to be detected.

In yet another embodiment, debubbling can be monitored using the vacuum level indicator of the fluid management system 22′. In semi-permeable cartridge-based degassing systems, the vacuum level used for degassing dips briefly every time a bubble flows through the degasser cartridge, as the vacuum pump labors or works to restore the vacuum to its previous level prior to the bubble(s) having compromised it as it flows through the cartridge. By monitoring these dips over a predetermined amount of time, the system is be able to detect when bubbles have ceased flowing (or at least below a predetermined level) through the degassing cartridge, and thus indicate that the debubbling step is complete.

In one embodiment, the positioning system is be outfitted with or includes a structure (such as a rubber-padded short rod) against which the probe assembly and/or the bolus assembly can be gently tapped to dislodge trapped gas or air bubbles. The location of this structure is known to the positioning system, and if this information is combined with the (known) physical characteristics of the probe and/or the bolus assembly (such as the size, orientation, location of interface/gripping mechanism, location of acoustic window, etc. of the probe and/or the bolus assembly), the structure can be used to define the tapping, manipulation, and other motions needed for dislodging the trapper gas or air bubbles and remove these from the bolus assembly.

5. Operation of One Embodiment

In one embodiment, the user chooses (from a selection of options) the required or desired transducer assembly, bolus assembly, and positioning assembly, as needed for the therapeutic ultrasound application. The fluid management system can already be connected to, or part of, the console assembly. The user can then connect or attach the positioning assembly to the console assembly, which configures the positioning assembly in a position amenable to the user to help with the bolus assembly and transducer assembly preparation (i.e., gripper mechanism extended, open, and/or horizontal).

The user can open a bolus assembly pack (which can be disposable or single-use), extract the bolus assembly, and attach a base of the bolus assembly to the positioning assembly via its gripper mechanism. In one embodiment, because of the mechanical structure of this connection, the base of the bolus assembly only fits into the gripper in one particular manner. This frees up the user's hands to place the selected transducer assembly into the bolus assembly (facilitated by the interconnect protection tip, as needed), and to finalize the assembly of the bolus by attaching an end cap and covering it with an acoustically transparent membrane, as needed. The positioning system can read the identifying information from the base of the bolus assembly, and can relay this information to the console assembly, which is able to start the appropriate ultrasound treatment software and set other now application-specific parameters needed for the correct operation of the system (such as expected bolus size, expected transducer assembly travel limits and degrees of freedom—translation/rotation/etc., bolus fluid volume, allowed acoustic membrane distention, etc.)

In one embodiment, the user can open a fluid path single-use kit that forms part of or attaches to the fluid management system 22′. Alternatively, disposable components of the fluid path could also be included in the bolus assembly pack. The user can connect the fluid path inlet/outlet tubings of the fluid management system to the bolus assembly (now fully sealed, and incorporating the transducer assembly), and can place the various components of the fluid management unit (e.g., peristaltic pump tubings, degasser cartridge, heat exchanger block, fluid reservoir, etc.) in the appropriate or desired locations.

Optionally, the fluid management system can automatically prime the fluid path. During this operation, the user can connect or attach the transducer assembly to the console assembly. The console assembly can read the identifying information of the transducer assembly, allowing the system to finalize its configuration, as now the specific positioning system, the bolus assembly, and the transducer assembly have been chosen and connected to the console. During this time, the user can also launch the application-specific ultrasound therapy software, from a list of possible options that are chosen by the system as being compatible with the selected components (e.g., the positioning assembly, the bolus assembly, and the transducer assembly).

In one embodiment, the system automatically debubbles the bolus assembly and/or the transducer assembly, and when complete, awaits for user input to place (automatically or under user guidance) the probe on/in the patient, in preparation for the treatment. If necessary, the user attaches the bolus assembly to the patient, as needed.

In one embodiment, the gripper mechanism(s) of the positioning system releases its grip from the bolus assembly, and attaches to the transducer assembly (automatically or under user guidance). This assembly is then placed on/in the patient at the location required for treatment. At this point, via small excursions of the transducer assembly as driven by the positioning system, and in combination with the ultrasound imaging function, the position, orientation, and/or travel extent of the transducer assembly within the bolus assembly is determined, guided by the mechanical bolus constraints/type identified by the system during the connection of the bolus assembly. After completing of this orientation and alignment step, the system places the transducer assembly within the bolus assembly in its ‘home’ position, allowing the user to start with the treatment planning procedure.

In one embodiment, the treatment is planned interactively by the user using image guidance, automatically by the system based on image features, and/or by incorporating additional information via multi-modality fusion techniques.

Optionally, the treatment can executed by the system via a coordinated interaction between (i) the positioning assembly mechanically positioning and/or orienting the transducer assembly to allow sonication of the target tissue, (ii) the electronic beam steering of the focal zone(s) of the therapy transducer to allow sonication of the target tissue, and/or (iii) the adjustment of the fluid volume within the bolus assembly, as all three activities or steps are able to influence the exact location and delivery of the ultrasound energy within the body. In one embodiment, prior to, during, and after treatment delivery, ultrasound image guidance is used to plan appropriate target locations for ultrasound delivery, monitor the progression of the treatment, and/or adjust the therapy execution due to expected (i.e. target tissue motion due to breathing or heartbeat), or unexpected tissue feedback and reaction to the ultrasound delivery (i.e. due to motion, heating, swelling, changes in blood flow, etc.).

In one embodiment (e.g., where the entire structure is held in place during the medical procedure by a body cavity (e.g., the rectum or a port)), upon treatment completion, the positioning system can release its grip from the transducer assembly, and can re-attach itself to the bolus assembly. In one embodiment, this can be done automatically, as the position of the gripper mechanism and/or the positioning system with respect to the bolus assembly is now known, due to initial bolus assembly/transducer assembly coordinate system registration step described above. The bolus assembly and the transducer assembly are removed from the patient, and placed in a comfortable user position/orientation for teardown, transducer assembly removal (by minimizing the changes of transducer assembly droppage), and/or cleanup. In another embodiment (e.g., where the entire structure is not held in place during the medical procedure by a body cavity), upon completion of the medical procedure, the probe assembly (i.e., the transducer assembly and the bolus assembly can be disconnected from the positioning assembly as a single unit. Then, the components of the probe assembly can be separated, cleaned and/or disposed of as necessary or desired. In yet another embodiment, two separate, spaced-apart and/or distinct positioning systems can be used as part of the system: one positioning system to manipulate or control the transducer assembly (e.g., dynamically), and another positioning system to hold or control the bolus assembly (e.g., statically).

In one embodiment, the fluid management system removes the fluid from the bolus assembly, allowing the user to remove the coupling membrane of the bolus assembly and/or disassemble the bolus assembly in order to extract from it the transducer assembly. The user disconnects the transducer assembly interconnect (or this can happen automatically when the gripper mechanism releases the transducer assembly), and removes the transducer assembly from the bolus assembly for disinfection, sterilization (for re-use), and/or disposal. In one embodiment, the bolus assembly is removed from the positioning system, and disposed.

6. Embodiments of Computer System

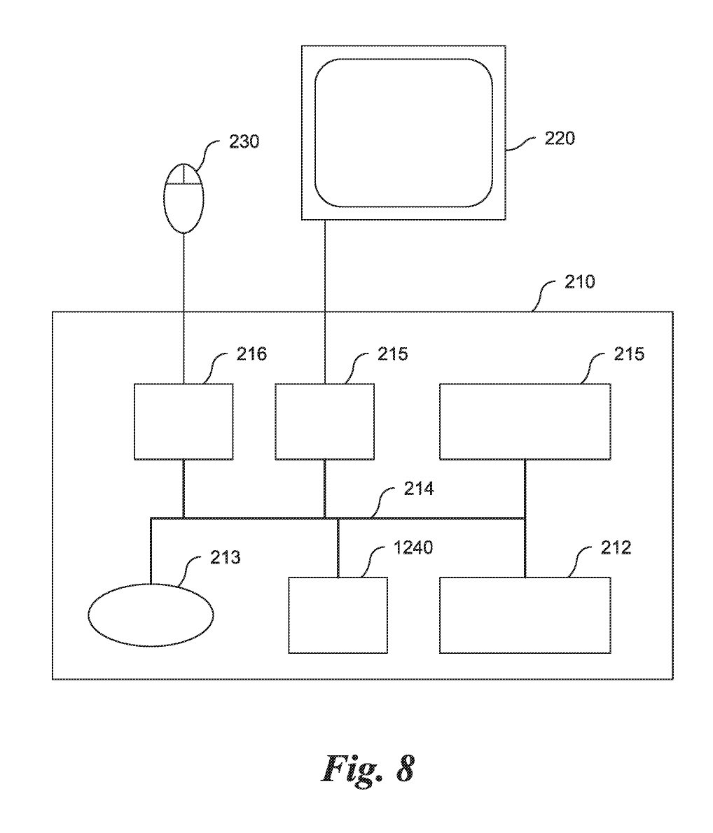

One or more of the above-described techniques and/or embodiments can be implemented with or involve software, for example modules executed on one or more computing devices 210 (see FIG. 8). Of course, modules described herein illustrate various functionalities and do not limit the structure or functionality of any embodiments. Rather, the functionality of various modules may be divided differently and performed by more or fewer modules according to various design considerations.

Each computing device 210 may include one or more processing devices 211 designed to process instructions, for example computer readable instructions (i.e., code), stored in a non-transient manner on one or more storage devices 213. By processing instructions, the processing device(s) 211 may perform one or more of the steps and/or functions disclosed herein. Each processing device may be real or virtual. In a multi-processing system, multiple processing units may execute computer-executable instructions to increase processing power.

The storage device(s) 213 may be any type of non-transitory storage device (e.g., an optical storage device, a magnetic storage device, a solid state storage device, etc.). The storage device(s) 213 may be removable or non-removable, and may include magnetic disks, magneto-optical disks, magnetic tapes or cassettes, CD-ROMs, CD-RWs, DVDs, BDs, SSDs, or any other medium which can be used to store information. Alternatively, instructions may be stored in one or more remote storage devices, for example storage devices accessed over a network or the internet.

Each computing device 210 additionally may have memory 212, one or more input controllers 216, one or more output controllers 215, and/or one or more communication connections 1240. The memory 212 may be volatile memory (e.g., registers, cache, RAM, etc.), non-volatile memory (e.g., ROM, EEPROM, flash memory, etc.), or some combination thereof. In at least one embodiment, the memory 212 may store software implementing described techniques.

An interconnection mechanism 214, such as a bus, controller or network, may operatively couple components of the computing device 210, including the processor(s) 211, the memory 212, the storage device(s) 213, the input controller(s) 216, the output controller(s) 215, the communication connection(s) 1240, and any other devices (e.g., network controllers, sound controllers, etc.). The output controller(s) 215 may be operatively coupled (e.g., via a wired or wireless connection) to one or more output devices 220 (e.g., a monitor, a television, a mobile device screen, a touch-display, a printer, a speaker, etc.) in such a fashion that the output controller(s) 215 can transform the display on the output device 220 (e.g., in response to modules executed). The input controller(s) 216 may be operatively coupled (e.g., via a wired or wireless connection) to one or more input devices 230 (e.g., a mouse, a keyboard, a touch-pad, a scroll-ball, a touch-display, a pen, a game controller, a voice input device, a scanning device, a digital camera, etc.) in such a fashion that input can be received from a user.

The communication connection(s) 1240 may enable communication over a communication medium to another computing entity. The communication medium conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

FIG. 8 illustrates the computing device 210, the output device 220, and the input device 230 as separate devices for ease of identification only. However, the computing device 210, the output device(s) 220, and/or the input device(s) 230 may be separate devices (e.g., a personal computer connected by wires to a monitor and mouse), may be integrated in a single device (e.g., a mobile device with a touch-display, such as a smartphone or a tablet), or any combination of devices (e.g., a computing device operatively coupled to a touch-screen display device, a plurality of computing devices attached to a single display device and input device, etc.). The computing device 210 may be one or more servers, for example a farm of networked servers, a clustered server environment, or a cloud service running on remote computing devices.

In one embodiment, the presently disclosed technology is directed to a non-transitory computer-readable medium having computer-readable code stored thereon that, when executed by one or more computing devices, causes the one or more computed devices to perform the one or more methods disclosed or claimed herein.

7. Recitation of Exemplary Embodiments

The following exemplary embodiments further describe optional aspects of the presently disclosed technology and are part of this Detailed Description. These exemplary embodiments are set forth in a format substantially akin to claims (each with numerical designations followed by the letter A), although they are not technically claims of the present application. The following exemplary embodiments refer to each other in dependent relationships as “embodiments” instead of “claims.”

1A. A system for delivering HIFU, the system comprising:

at least one positioning assembly operatively connected to a console assembly having a computer system, the at least one positioning assembly including a robotic arm movable upon receipt of one or more instructions from the computer system, the at least one positioning assembly including at least one gripper mechanism configured to selective grip and release an object;

at least one ultrasound probe assembly including an ultrasound transducer assembly and a bolus assembly, at least a portion of the ultrasound transducer assembly being removably positionable within the bolus assembly, at least the ultrasound transducer assembly of the probe assembly being selectively connected to gripper mechanism and moved by the positioning assembly.

2A. The system of embodiment 1A, wherein at least a portion of a shaft of the ultrasound transducer assembly has a larger diameter than a diameter of an interconnect mechanism of the ultrasound transducer assembly to facilitate connecting the ultrasound transducer assembly with the bolus assembly.

3A. The system of embodiment 1A, wherein the computer system is configured to manipulate the positioning assembly to remove at least some gas bubbles from fluid circulated in at least one of the bolus assembly and the transducer assembly.

4A. The system of embodiment 1A, further comprising:

a computer system including:
one or more processors; and
one or more memories operatively coupled to the one or more processors and having computer readable instructions stored thereon which, when executed by at least one of the one or more processors, causes the at least one of the one or more processors to instruct the positioning assembly to change its configuration or position.

5A. A system for delivering HIFU, the system comprising:

one or more processors; and

one or more memories operatively coupled to the one or more processors and having computer readable instructions stored thereon which, when executed by at least one of the one or more processors, causes the at least one of the one or more processors to:

a) transmit one of light and ultrasound energy through a fluid conduit connected to at least one of a transducer assembly and a bolus assembly of an ultrasound probe assembly, the light or ultrasound energy being emitted from a first side of the fluid conduit;

b) receive any of the light or ultrasound energy that travels through the fluid conduit at an opposing second side of the fluid conduit;

c) detect whether the light or ultrasound energy has been disrupted when traveling through the fluid conduit; and

e) transmit a signal when it is determined that the light or ultrasound energy has not been disrupted when traveling through the fluid conduit, the signal indicating that no bubbles or a limited number of bubbles has been detected in the fluid conduit.

6A. The system of embodiment 1A, further comprising:

a light source positioned on or near a side of the first tube; and

a detector positioned on or near an opposing side of the first tube, the detector being operatively connected to the computer system,

wherein a gas bubble flowing through the first tube disrupts a flow of light from the light source to the detector, thereby indicating to the computer system the presence of at least one gas bubble in the fluid circulated in at least one of the bolus assemblies and the transducer assembly.

7A. A system for degassing a bolus assembly of a high intensity focused ultrasound probe assembly, the system comprising:

a high intensity focused ultrasound probe assembly including a transducer assembly and a bolus assembly;

one or more processors; and

one or more memories operatively coupled to the one or more processors and having computer readable instructions stored thereon which, when executed by at least one of the one or more processors, causes the at least one of the one or more processors to:

transmit one of light and ultrasound energy from a source through a fluid conduit connected to at least one of the transducer assembly and the bolus assembly, the light or ultrasound energy being emitted from a first side of the fluid conduit;

receive any of the light or ultrasound energy that travels from the source through the fluid conduit at an opposing second side of the fluid conduit;

detect whether the light or ultrasound energy has been disrupted when traveling through the fluid conduit;

repeat the above steps when it is determined that the light or ultrasound energy has been disrupted when traveling through the fluid conduit; and

transmit a signal to at least one of a display and a speaker when it is determined that the light or ultrasound energy has not been disrupted when traveling through the fluid conduit, the signal indicating that no bubbles or a predetermined amount of bubbles has been detected in the fluid conduit.

8A. The system of embodiment 7A, wherein the source forms part of the ultrasound transducer array.

9A. The system of embodiment 7A, wherein the source is an ultrasound transducer.

10A. A system for degassing a bolus assembly of a high intensity focused ultrasound probe assembly, the system comprising:

a high intensity focused ultrasound probe assembly including a transducer assembly and a bolus assembly;

one or more processors; and

one or more memories operatively coupled to the one or more processors and having computer readable instructions stored thereon which, when executed by at least one of the one or more processors, causes the at least one of the one or more processors to:

a) transmit one of light and ultrasound energy through a fluid conduit connected to at least one of the transducer assembly and the bolus assembly, the light or ultrasound energy being emitted from a first side of the fluid conduit;

b) receive any of the light or ultrasound energy that travels through the fluid conduit at an opposing second side of the fluid conduit;

c) detect whether the light or ultrasound energy has been disrupted when traveling through the fluid conduit;

d) repeat steps a) and b) when it is determined that the light or ultrasound energy has been disrupted when traveling through the fluid conduit; and

e) transmit a signal to at least one of a display and a speaker when it is determined that the light or ultrasound energy has not been disrupted when traveling through the fluid conduit, the signal indicating that no bubbles or a predetermined amount of bubbles has been detected in the fluid conduit.

The system of embodiment 10A, wherein the light is emitted from a light emitting diode.

The above disclosed systems, apparatuses, methods and description of generic embodiments of the presently disclosed technology are provided to enable any person skilled in the art to make or use the invention. Various modifications to the embodiments described herein will be readily apparent to those skilled in the art, and the generic principles described herein can be applied to other embodiments without departing from the spirit or scope of the presently disclosed technology. Thus, it is to be understood that the description and drawings presented herein represent a functional generic embodiment of the presently disclosed technology and are, therefore, representative of the subject matter which is broadly contemplated by the presently disclosed technology. It is further understood that the scope of the presently disclosed technology fully encompasses other embodiments that may become obvious to those skilled in the art and that the scope of the presently disclosed technology is accordingly limited by nothing other than the appended claims.

I/We claim:

1. An ultrasound delivery system comprising:
a console assembly including a display and a computer system;
a positioning assembly operatively connected to the console assembly, the positioning assembly being articulable between a plurality of different configurations upon receipt of one or more instructions from the computer system, the positioning assembly including at least one gripper mechanism configured to selective grip and release; and
at least one probe assembly including an ultrasound transducer assembly, a first bolus assembly and a second bolus assembly, the first bolus assembly having at least a different geometry than the second bolus assembly, at least a portion of the ultrasound transducer assembly being removably positionable within each of the first bolus assembly and the second bolus assembly, at least a portion of the probe assembly being selectively connected to gripper mechanism and moved by the positioning assembly.

2. The system of claim 1, wherein the at least one gripper mechanism is configured to selectively engage and release at least a portion of the ultrasound transducer assembly.

3. The system of claim 1, wherein the at least one gripper mechanism includes a first gripper mechanism and a second gripper mechanism, the first gripper mechanism being configured to selectively connect to at least a portion of the ultrasound transducer assembly, the second gripper mechanism being configured to selectively connect to at least a portion of the bolus assembly.

4. The system of claim 1, wherein the ultrasound transducer assembly includes a shaft, at least one transducer positioned at or near a distal end of the shaft, and an interconnect mechanism positioned at or near a proximal end of the shaft, the interconnect mechanism being configured to electrically connect the transducer assembly to the console assembly via the positioning assembly.

5. The system of claim 4, wherein at least a portion the shaft has a larger diameter than a diameter of the interconnect mechanism to facilitate selectively connecting the transducer assembly with the first bolus assembly and the second bolus assembly.

6. The system of claim 4, wherein the ultrasound transducer assembly includes at least one keyed attachment on at least one of the shaft and the interconnect mechanism, the at least one keyed attachment being selected from the group consisting of a groove extending parallel to a longitudinal axis of the shaft, a projection extending parallel to a longitudinal axis of the shaft, and a pyramid structure.

7. The system of claim 1, wherein the at least one probe assembly includes a first probe assembly and a second probe assembly, the second probe assembly being configured to utilize the same ultrasound transducer assembly as the first probe assembly, the second probe assembly being configured to utilize a different bolus assembly than the first probe assembly.

8. The system of claim 1, wherein the positioning assembly is operatively connected to the console assembly by an electrical connection, the electrical connection being selected from the group consisting of a network interface, a USB interface, and a wireless connection.

9. The system of claim 1, wherein the computer system is configured to receive ultrasound image guidance from the transducer assembly and adjust the position of at least one of the transducer assembly and the bolus assembly.

10. The system of claim 1, wherein the positioning assembly is a robotic arm.

11. The system of claim 1, wherein transducer assembly includes or contains an identifier to enable the computer system to identify at least one of the presence of and connection to the transducer assembly, the identifier being selected from the group consisting of a non-volatile memory that contains at least one of a serial number, a barcode and a unique classification, one or more dimples on the interconnect mechanism, and one or more bumps on the interconnect mechanism.

12. The system of claim 1, further comprising:
a fluid management system supported or enclosed by the console assembly, the fluid management system being fluidly connected to at least one of the first bolus assembly, the second bolus assembly, and the ultrasound transducer assembly, the fluid management system being configured to circulate fluid within at least one of the bolus assemblies and the ultrasound transducer assembly through a first tube connecting the fluid management system to a first fluid port of one of the bolus assemblies and the transducer assembly and from a second tube connecting a second fluid port of one of the bolus assemblies and the transducer assembly to the fluid management system,
wherein the computer system is configured to manipulate the positioning assembly to remove at least some gas bubbles from the fluid circulated in at least one of the bolus assemblies and the transducer assembly.

13. The system of claim 12, wherein the ultrasound transducer assembly includes at least one imagining transducer selectively positionable within a bolus of each of the first and second bolus assemblies, and wherein the system further comprises:
one or more processors; and
one or more memories operatively coupled to the one or more processors and having computer readable instructions stored thereon which, when executed by at least one of the one or more processors, causes the at least one of the one or more processors to create a map of at least a portion of an interior of the bolus of at least one of the first and second bolus assemblies.

14. The system of claim 12, further comprising:
an ultrasound transmitter positioned on or near a side of the first tube; and
an ultrasound detector positioned on or near an opposing side of the first tube, the ultrasound detector being operatively connected to the computer system,
wherein a gas bubble flowing through the first tube disrupts a flow of ultrasound energy from the ultrasound transmitter to the ultrasound detector, thereby indicating to the computer system the presence of at least one gas bubble in the fluid circulated in at least one of the bolus assemblies and the transducer assembly.

15. The system of claim 1, wherein the computer system, based on one or more signals received from one or more detectors or sensors, is configured to instruct the positioning assembly to move from a first configuration to a second configuration, thereby removing at least some gas bubbles from fluid circulated in at least one of the first bolus assembly, the second bolus assembly, and the transducer assembly.

16. A system for degassing a bolus assembly of a high intensity focused ultrasound probe assembly, the system comprising:
a high intensity focused ultrasound probe assembly including a transducer assembly and a bolus assembly, the transducer assembly including at least one transducer, the at least one transducer being configured to deliver ultrasound therapy and generate one or more ultrasound images;
one or more processors; and
one or more memories operatively coupled to the one or more processors and having computer readable instructions stored thereon which, when executed by at least one of the one or more processors, causes the at least one of the one or more processors to:
a) transmit one of light and ultrasound energy through a fluid conduit connected to at least one of the transducer assembly and the bolus assembly, the light or ultrasound energy being emitted from a first side of the fluid conduit;
b) receive any of the light or ultrasound energy that travels through the fluid conduit at an opposing second side of the fluid conduit;
c) detect whether the light or ultrasound energy has been disrupted when traveling through the fluid conduit;
d) repeat steps a) and b) when it is determined that the light or ultrasound energy has been disrupted when traveling through the fluid conduit; and
e) transmit a signal to at least one of a display and a speaker when it is determined that the light or ultrasound energy has not been disrupted when traveling through the fluid conduit, the signal indicating that no bubbles or less than a predetermined amount of bubbles has been detected in the fluid conduit.

17. The system of claim 16, wherein at least a portion of the ultrasound transducer assembly is removably positionable within the bolus assembly.

18. The system of claim 16, further comprising:
a console assembly including the display, the speaker, the one or more processors, and the one or more memories; and
a positioning assembly operatively connected to the console assembly, the positioning assembly being articulable between a plurality of different configurations upon receipt of one or more instructions from the computer system, the positioning assembly including at least one gripper mechanism configured to selective grip and release; and
wherein at least a portion of the ultrasound transducer assembly is removably positionable within the bolus assembly, and wherein at least a portion of the probe assembly is selectively connected to gripper mechanism and moved by the positioning assembly.

19. A system for degassing a bolus assembly of a high intensity focused ultrasound probe assembly, the system comprising:
a high intensity focused ultrasound probe assembly including a transducer assembly and a bolus assembly, the transducer assembly including at least one transducer, the at least one transducer being configured to deliver ultrasound therapy and generate one or more ultrasound images;
one or more processors; and
one or more memories operatively coupled to the one or more processors and having computer readable instructions stored thereon which, when executed by at least one of the one or more processors, causes the at least one of the one or more processors to generate a 2D or 3D ultrasound image of an inside region of the bolus assembly, the ultrasound image being configured to display any fluid and any bubbles of a predetermined size or greater in the inside region of the bolus assembly.

20. The system of claim 19, wherein the computer readable instructions, when executed by at least one of the one or more processors, causes the at least one of the one or more processors to transmit a signal to at least one of a display and a speaker when it is determined that no bubbles or less than a predetermined amount of bubbles has been detected in the fluid conduit.

21. The system of claim 19, further comprising:
a console assembly including the display, the speaker, the one or more processors, and the one or more memories; and
a positioning assembly operatively connected to the console assembly, the positioning assembly being articulable between a plurality of different configurations upon receipt of one or more instructions from the computer system, the positioning assembly including at least one gripper mechanism configured to selectively grip and release; and
wherein at least a portion of the ultrasound transducer assembly is removably positionable within the bolus assembly, and wherein at least a portion of the probe assembly is selectively connected to gripper mechanism and moved by the positioning assembly.
